(19) United States
(12) Patent Application Publication
(10) Pub. No.: US 2007/0071793 A1
(43) Pub. Date: Mar. 29, 2007

(54) Cure for prostate enlargement frequency of urination and cancer

(76) Inventor: Leon Clark, Tulsa, OK (US)

Correspondence Address: Leon Clark, 5642 N. Frankfort Ave., Tulsa, OK, 74126, US

(21) Appl. No.: 11/199,436

(22) Filed: Sep. 23, 2005

(51) Int. Cl. A61K 47/00

(52) U.S. Cl. 424/439

(57) ABSTRACT

This is a chemical compound designed to eradicate cancer from the prostate (and possibly other parts of the body) restore the prostate to it's normal size, cure frequency of urination and restore urination back to it's youthful state.

This is a chemical compound made by mixing 4 ozs. of The Palma Christi Castor Oil, 1 level tsp. of baking soda (Arm & Hammer) 1 pinch of salt and 1 tsp. of Cod-liver oil.

Place the components into a 4.5 oz. enema bottle and shake until well mixed. Expel the air contents from the bottle by squeezing the bottle until the components oozes from the tip. Lubricate the tip of the bottle and the index finger (your choice) with the oozed out portion. While still holding pressure on the bottle, insert the enema tube into the rectum and apply pressure until about 1/7th of mixture is expelled into the rectum. While still holding pressure (not squeezing) on the bottle, pull the tube out. This will prevent seepage back into the bottle and defiling of the remaining contents. Insert the lubricated finger into the rectum and message the prostate while contracting and releasing pressure on it (prostate). Next, hold the bottle over the sink and apply slight pressure to expel any contaminants that may have entered the tip end. Run hot water over the tip and wash with soap. Recap and store in bathroom for usage the next day. If no bowl-movements, skip the next day and repeat the following day. Place folded tissue on the rectum between the cheeks of the hip for absorption in case of accidental expulsion. The mixture should be retained in the rectum unless bowl-movement dictates otherwise, discomfort occurs or one has to leave the pre-misses. If that is the case, expulge and repeat the process the next day.

1. I claim a cure for prostate cancer (and other cancers such as uterine and ovarian-follow same procedure outlined in specification). The cure is composed of the following components:
1.) 4 ozs. of The Palma Christi Castor Oil
2.) 1 level tsp. of baking soda (Arm & Hammer)
3.) 1 pinch of salt
4.) 1 tsp. of cod-liver oil
I further claim a cure for prostate enlargement and frequency of urination in this—my aforementioned claim.
